(19) United States
(12) Patent Application Publication
(10) Pub. No.: US 2026/0049316 A1
(43) Pub. Date: Feb. 19, 2026

(54) ANTISENSE OLIGONUCLEOTIDE FOR INHIBITING ACSL4 GENE EXPRESSION AND ITS APPLICATIONS

(71) Applicant: Yunnan University, Kunming (CN)

(72) Inventors: Dahua Chen, Kunming (CN); Yuanxiang Zhu, Kunming (CN)

(21) Appl. No.: 19/200,094

(22) Filed: May 6, 2025

(30) Foreign Application Priority Data
Jun. 12, 2024 (CN) 202410749135.1

(51) Int. Cl. C12N 15/113

(52) U.S. Cl. CPC C12N 15/1137; C12N 2310/11

(57) ABSTRACT

The present invention provides antisense oligonucleotides (ASO) for inhibiting ACSL4 gene expression. The oligonucleotide sequences are represented by SEQ ID NO.3 and SEQ ID NO.7. The antisense oligonucleotide sequences are fully phosphorothioated, and the ribose of the initial and termina five bases at each end is modified with a 2′-O-Methoxyethyl group. Additionally, all cytosines are modified with methylcytosine. Cell experiments and animal model studies conducted in this invention have demonstrated that the ACSLA ASO can effectively alleviate the ferroptosis damage inflicted on neural cells during the process of stroke. Moreover, modulation of ACSLA expression is beneficial for a range of medical disorders including acute ischemic strokes, cancer-related diseases, metabolic disorders, neurodegenerative diseases and ferroptosis related diseases, thus presenting a bright future for clinical utilization.

### CROSS-REFERENCE TO RELATED APPLICATIONS

This application claims priority to Chinese Patent Application No. 202410749135.1, filed on Jun. 12, 2024, which is hereby incorporated by reference in its entirety.

### TECHNICAL FIELD

This invention belongs to the field of biotechnology and biopharmaceuticals, specifically providing antisense oligonucleotide for inhibiting ACSL4 gene expression and its applications. Reduction of ACSL4 protein expression is beneficial for a range of ACSL4 dependent diseases, such as cancer, metabolic diseases and Ischemia-reperfusion injury.

### SEQUENCE LISTING

The present application contains a sequence listing which was filed electronically in XML format and is hereby incorporated by reference in its entirety. Besides, the XML copy is created on Nov. 7, 2025, is named “Antisense Oligonucleotide for Inhibiting ACSL4 Gene Expression and Its Applications-Sequence Listing” and is 10,006 bytes in sizes.

### BACKGROUND

Cerebral stroke remains one of the leading causes of mortality and disability in modern society. It refers to the interruption of blood flow supply to the brain due to ischemia or hemorrhage in cerebral vessels, ultimately leading to brain tissue damage. Ischemic stroke (IS) accounts for approximately 85% of all strokes. Current research on drugs for IS primarily focuses on thrombolysis and neuroprotection. Clinical studies have shown that dissolving thrombi to restore blood flow perfusion within 4.5 hours of the onset of ischemia can save brain neurons that are on the verge of death. However, using these drugs 4.5-6 hours after ischemia can exacerbate pathological injuries due to ischemia-reperfusion, leading to increased mortality. Therefore, the use of thrombolytic drugs is strictly time-limited. Neuroprotection was once considered a promising approach for IS treatment, aiming to protect brain cells from damage. However, to date, no independent neuroprotective agent has been clinically proven effective. Consequently, finding effective therapeutic strategies and drugs is of utmost importance.

Recent studies have revealed that ferroptosis, a regulated form of cell death, plays a pivotal role in mediating neuronal cell death during ischemic stroke (IS). First identified in 2012, ferroptosis is primarily driven by the toxic accumulation of lipid peroxides within the cell membrane. This process involves the inhibition of antioxidant defense systems and the accumulation of iron-dependent reactive oxygen species (ROS), which react with polyunsaturated fatty acids (PUFAs) and disrupt the integrity of the cell membrane. The hallmark of ferroptosis is the accumulation of peroxidized PUFAs in the cell membrane. Ferroptosis is one of the most critical forms of cell death during brain ischemia-reperfusion injury and has been shown to participate in the ischemia-reperfusion process. Thus, targeting ferroptosis post-IS is a promising therapeutic approach.

The ACSL4 gene, known as Acyl-CoA Synthetase Long-Chain Family Member 4,encodes an enzyme that catalyzes the formation of long-chain fatty acyl-CoA by combining long-chain fatty acids with coenzyme A (CoA). Long-chain fatty acyl-CoA is a key intermediate in lipid metabolism and the synthesis of biological membranes. ACSL4 increases the content of PUFAs in the cell membrane by catalyzing the formation of fatty acyl-CoA, thereby increasing the cell's sensitivity to ferroptosis. Additionally, the expression level of ACSL4 may affect the activity of the cellular antioxidant defense system, including antioxidant substances such as glutathione (GSH) and related enzymatic systems. When the antioxidant defense system is inhibited, the peroxidation of PUFAs in the membrane cannot be effectively suppressed, thus exacerbating ferroptosis. Therefore, the expression and activity of ACSL4 are closely related to ferroptosis sensitivity. High expression or activity of ACSL4 increases the cell's susceptibility to ferroptosis, while inhibiting ACSL4 expression or activity can protect cells from ferroptosis. Given this, developing inhibitors for ACSL4 is highly significant for reducing neuronal damage in ischemic stroke.

### OBJECTIVE OF THE INVENTION

The present invention provides ASOs which can inhibit ACSL4 expression and the use of ASOs to treat or prevent diseases related to the functioning of the ACSL4. The invention designed 10 ASOs targeted ACSL4 pre-mRNA. Furthermore, the invention introduces optimized ASO sequences and chemical modifications that significantly reduce ACSL4 levels, thereby inhibiting ferroptosis and mitigating neuronal damage in rodent stroke models. These advancements highlight the therapeutic potential of the ASOs in addressing ACSL4-related pathologies.

### SUMMARY

To address the issues mentioned in the background, the present invention designed ASOs targeting ACSL4 pre-mRNA represented by SEQ ID NO: 1 to 10. The use of the oligonucleotide to treat or prevent diseases related to the functioning of the ACSL4.

ASOs are artificially synthesized, chemically modified single-stranded oligonucleotides designed to specifically bind to complementary sequences on target RNA. Upon binding, ASOs can modulate the activity of the target RNA through various mechanisms. The most common mode of action involves the recruitment of endogenous RNase H1, an enzyme that specifically cleaves the target RNA. After cleavage, the ASO is released and can bind to additional target RNA molecules, thereby achieving sustained inhibition of the target gene. To enhance their stability within the body and prevent rapid degradation, ASOs undergo chemical modifications. These modifications not only prolong their lifespan but also improve their efficacy. ASO drugs offer several advantages, including high specificity, diverse mechanisms of action, broad applicability, low immunogenicity, minimal toxicity and side effects, and a relatively short development cycle. These attributes make ASOs a highly promising and preferred therapeutic approach for treating a wide range of diseases.

Selectively, the gene fragment is the sequence of SEQ ID No.3 and SEQ ID No.7.The sequence and modifications of SEQ ID NO.3 are as follows:

(MOE-T)*(MOE-G)*(MOE-G)*(MOE-G)*(MOE-5-Me-C)*(dT)*(dT)*(m5dC)*
(dA)*(dG)*(dT)*(dA)*(m5dC)*(dA)*(dG)*(MOE-T)*(MOE-A)*(MOE-5-Me-C)*(MOE-
A)*(MOE-G);

The sequence of SEQ ID NO.7 is as follows:

(MOE-G)*(MOE-5-Me-C)*(MOE-A)*(MOE-G)*(MOE-A)*(m5dC)*(dA)*(dT)
*(dT)*(m5dC)*(dA)*(dT)*(dG)*(dA)*(dA)*(MOE-T)*(MOE-5-Me-C)*(MOE-G)*(MOE-
G)*(MOE-T);

Preferably, the antisense oligonucleotide sequences are fully phosphorothioated (PS, indicated by *), and the ribose of the first and last five bases at each end is modified with 2′-O-methoxyethyl (2′-MOE). All cytosines are methylated.

Preferably, the antisense oligonucleotide sequences undergo other modifications, including LNA (Locked Nucleic Acid), tricyclic DNA, 2′-O-methyl, 2′-O-methoxyethyl (MOE), UNA (Unlocked Nucleic Acid), 2′-fluoro, and conformationally restricted nucleosides.

This invention designs 10 antisense oligonucleotides targeting the full length of ACSL4 based on the predicted RNA structure of ACSL4. The antisense oligonucleotides provided by this invention show clear specificity and effectiveness in inhibiting ACSL4.

TABLE 1

| SEQ ID No. | Sequences (5′-3′) |
|---|---|
| SEQ ID No. 1 | TGTACTCACCGGAACAGCAG |
| SEQ ID No. 2 | GTACTCACCGGAACAGCAGC |
| SEQ ID No. 3 | TGGGCTTCAGTACAGTACAG |
| SEQ ID No. 4 | TTCAGTACAGTACAGTCTCC |
| SEQ ID No. 5 | TACTCACCGGAACAGCAGCC |
| SEQ ID No. 6 | TCCCAGCACCACATGATTCT |
| SEQ ID No. 7 | GCAGACATTCATGAATCGGT |
| SEQ ID No. 8 | CCAGCACCACATGATTCTGT |
| SEQ ID No. 9 | CAGACATTCATGAATCGGTG |
| SEQ ID No. 10 | CATTCATGAATCGGTGTGTC |

The modified ACSL4 antisense oligonucleotides provided by this invention mainly include the addition of specific chemical modifications to the original antisense oligonucleotide sequences targeting ACSL4, making these sequences more stable and suitable for cell transfection experiments and commercial applications. The chemical modifications to the 10 antisense oligonucleotide sequences provided by this invention primarily involve the following specific types of modifications:
1. Phosphorothioate Bond Modification: This refers to the replacement of one oxygen atom in the phosphodiester bond (PO) of each nucleotide in the ASO molecule with a sulfur atom (PS). This modification enhances the stability of ASOs against nucleases and increases their affinity for protein binding.
2. 2′-O-Methoxyethyl Group Modification: This involves the modification of the 2′-O-methoxyethyl (MOE) group in the 5′ and 3′ end of five consecutive nucleotides in the ASO molecule. The 2′-O-methoxyethyl group is a modification where the 2′ position of the furanose ring is 3-methoxy-ethyl (the hydroxyl group is modified to a methoxyethyl group). This modification enhances nuclease resistance, reduces off-target effects, and increases the hybridization affinity of antisense oligonucleotides.
3. 5-Methyldeoxycytidine (5-Me-dC) Modification: This involves the modification of a methyl group at the fifth position of the cytosine deoxynucleotide base in the ASO molecule. 5-Me-dC pairs with dG, and this modification increases the stability of the double strand and significantly weakens the immune response.

The results of Q-PCR and Western Blot after transfecting ASOs into cells show that ASO-3 and ASO-7 can effectively knock down the expression of ACSL4 protein. ASO-7 has a better knockdown effect, with a reduction of over 80% at 200 nM. This invention also provides results from animal model experiments, demonstrating that knocking down ACSL4 in the rat brain can reduce the infarct area in rats with ischemia-reperfusion injury.

Compared to existing technologies, this invention has the following beneficial effects:
The cell experiments and animal model experiments provided by this invention prove that ACSL4 antisense oligonucleotides can effectively alleviate neuronal damage during the stroke process. Moreover, the antisense oligonucleotides provided by this invention, as a new type of nucleic acid drug, have broad application prospects for the adjuvant treatment of stroke.

### BRIEF DESCRIPTION OF DRAWINGS

FIG. 1: Distribution of the ASOs on the ACSL4 gene loci.

FIG. 2: Q-PCR results showing the changes of ACSL4 mRNA levels 24 hours after ASO transfection in 293T cells.

FIG. 3: Q-PCR results showing the changes of ACSL4 mRNA levels 36 hours after ASO-3, ASO-7, and ASO-10 transfection in 293T cells.

FIG. 4: Western Blot results showing the changes of ACSL4 protein levels 36hours after ASO-3 and ASO-7 transfection in 293T cells.

FIG. 5: Schematic diagram showing the reduction in MDA content due to ferroptosis after ASO-7 transfection in 293T cells.

FIG. 6: Neurobehavioral score graph showing that ACSL4-ASO7 can alleviate the behavioral changes caused by ferroptosis-induced neuronal damage; where, Sham: sham operation group, MCAO/R: model group, ### indicates the model group, ** indicates the ASO-treated model group, model group compared to sham operation group, P<0.001; ** indicates the ASO-treated model group compared to the untreated model group, P<0.01.

FIG. 7: (Triphenyl Tetrazolium Chloride) TCC results showing that ACSL4-ASO7 can reduce the infarct volume in the rat model of cerebral ischemia-reperfusion injury; where, Sham: sham operation group, MCAO/R: model group, ### indicates the model group, ** indicates the ASO-treated model group, model group compared to sham operation group, P <0.001; ** indicates the ASO-treated model group compared to the untreated model group, P <0.05.

FIG. 8: HE staining results showing that ACSL4-ASO7 can reduce the infarct volume in a rat model of cerebral ischemia-reperfusion injury; where, Sham: sham operation group, MCAO/R: model group, ### indicates the model group, ** indicates the ASO-treated model group, model group compared to sham operation group, P<0.001; ** indicates the ASO-treated model group compared to the untreated model group, P<0.05.

### DESCRIPTION OF EMBODIMENTS

The following detailed descriptions are provided in conjunction with the figures and examples to illustrate the implementation methods of this invention. These examples are intended to describe the invention but are not to be construed as limiting its scope.

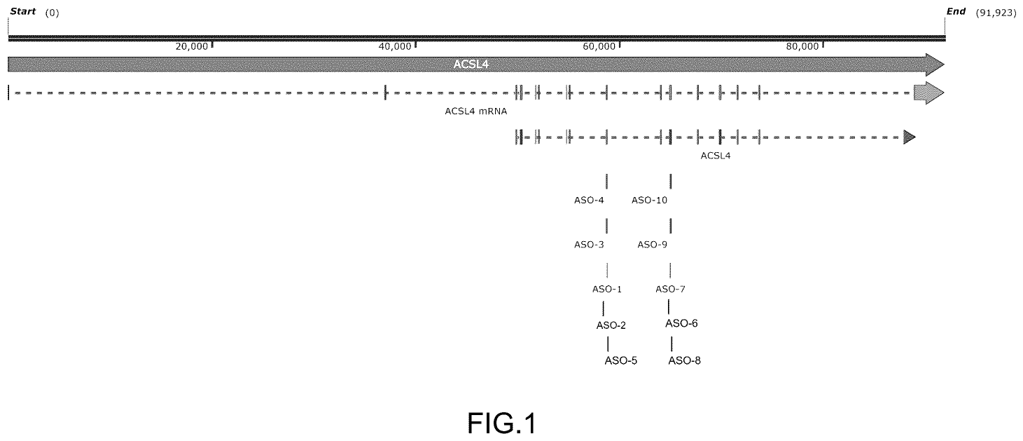

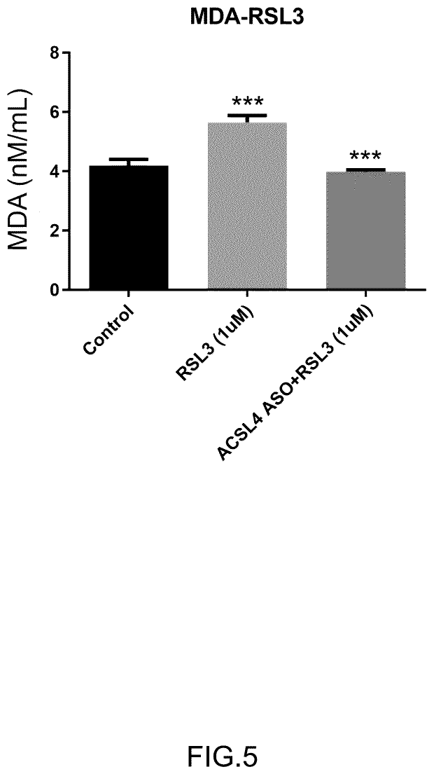

As shown in FIGS. 1 to 8:

Example 1: Transfection of ASOs in HEK293 cells and detection of Acs14 mRNA, with the specific steps as follows:
1. Cell Culture: HEK293 cells were cultured in DMEM medium containing 10% fetal bovine serum (FBS). HepG2.2.15 cells were cultured in MEM medium containing 10% FBS. Cells were seeded 24 hours before transfection at a density of 2.5×10\hat{ }4 cells per well in a 12-well cell culture plate. The next step was performed when the cells reached 70%-80% confluence.
2. Cell Transfection: The appropriate amount of ASO was added to 50 μL of Opti-MEM and mixed thoroughly to form Solution A. An appropriate amount of Celopener transfection reagent was added to 50 μL of Opti-MEM and mixed thoroughly to form Solution B. Solution A was added dropwise to Solution B, mixed thoroughly, and the mixture was incubated at room temperature for 30 minutes. The mixture was then added dropwise to the wells containing the cells, and the cells were cultured in a CO2 incubator at 37° C.
3. 8 Hours Post-Transfection: The cell culture medium containing the transfection reagent was removed, and 1 mL of complete medium was added to each well.
4. 48 Hours Post-Transfection: The cell culture medium was removed, and 500 μL of pre-chilled PBS buffer was added to each well to wash away residual medium, which was then removed.
5. 500 μL of Trizol Lysis Reagent was added to each well. After the cells were fully lysed, the lysis solution was transferred to a 1.5 mL nuclease-free EP tube, and 100 μL of chloroform was added. The mixture was vortexed thoroughly.
6. Incubation at Room Temperature for 3 Minutes, followed by centrifugation at 12000 g for 15 minutes at 4° C.
7. Centrifugation Complete: 200 μL of the upper layer was transferred to a new 1.5 mL nuclease-free EP tube, and an equal volume of isopropanol was added. The mixture was vortexed thoroughly and centrifuged at 12000 g for 15 minutes at 4° C.
8. Retain Precipitate: Remove the supernatant and wash the precipitate twice with 75% ethanol.
9. Washing Complete: Remove 75% ethanol thoroughly, leaving only the precipitate, and air-dry at room temperature for 5 minutes.
10. Dissolve RNA with Nuclease-Free Water and measure the RNA concentration using a nanodrop 2000.
11. Reverse Transcription: 1 ug of RNA was used for reverse transcription with a total reaction volume of 20 μL. After the reverse transcription reaction, 80 μL of nuclease-free water was added to dilute the cDNA.
12. Preparation of the Following Mixture in a PCR Tube

| | | |
|---|---|---|
| 2 × Hieff ® qPCR SYBR ® Green Master Mix | 10.0 | μl; |
| Primer 1 (10 μM) | 0.4 | μl, |
| Primer 2 (10 μM) | 0.4 | μl |
| Template cDNA | x | μl |

| | |
|---|---|
| ddH2O | up tp 20.0 μl |

qPCR Internal Reference Primers for Human GAPDH: Forward primer sequence as shown in Seq ID No. 11: 5′-GGAGCGAGATCCCTCCAAAAT-3′, Reverse primer sequence as shown in Seq ID No. 12: 5′-GGCTGTTGTCATACTTCTCATGG-3′, Primers for Detecting the ACSL4 Gene: Forward primer sequence as shown in Seq ID No. 13: 5′-CATCCCTGGAGCAGATACTCT-3′, Reverse primer sequence as shown in Seq ID No. 14: 5′-TCACTTAGGATTTCCCTGGTCC-3′

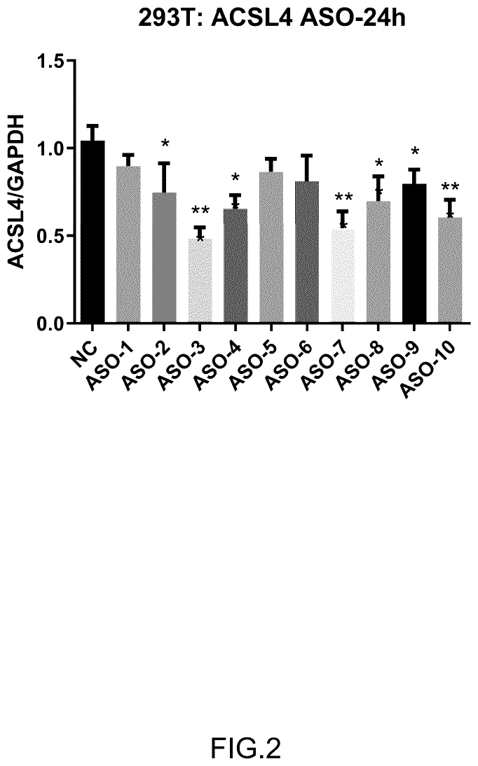

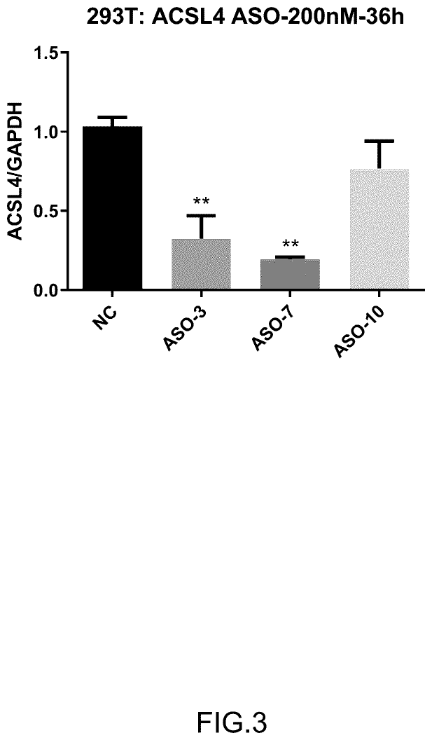

As Shown in FIGS. 2 and 3: FIG. 2 shows the ACSL4 mRNA levels in HEK293 cells 24 hours after transfection with 100 nM ACSL4 ASO. Among the ASOs, ASO-3 and ASO-7 have the best knockdown effects, with a knockdown efficiency of over 50% at 100 nM.

FIG. 3 shows the knockdown effects of ASO-3 and ASO-7 36 hours after transfection with 200 nM. The knockdown efficiency can reach 70% and 80%, respectively.

Example 2: Culturing of HEK293 Cells, ASO Transfection, and Detection of ACSL4 Protein, the steps are as follows:
1. Cell Culture and Transfection: Follow the steps described above.
2. Cell Harvesting and Lysis

Cell Harvesting: Collect the cells using a cell scraper 24 or 48 hours after transfection.

Cell Lysis: Use 200 μL of lysis buffer (add protease inhibitors at a 1:100 ratio just before use).
3. Protein Concentration Determination: Measure the protein concentration of the samples using the BCA protein assay method. Adjust the protein loading volume based on the measured concentration.
4. Sample Preparation for Loading: Mix the cell lysate with SDS sample buffer and heat at 95° C. for 5 to 10 minutes to denature the proteins. Load the samples onto an SDS-PAGE gel and perform electrophoresis using electrophoresis buffer.
5. Blotting: Transfer the proteins separated by electrophoresis from the gel to a polyvinylidene fluoride (PVDF) membrane.
6. Blocking: Incubate the membrane with blocking buffer (5% skim milk dissolved in PBST) at room temperature for 1 hour to reduce non-specific binding.
7. Primary Antibody Incubation: Incubate the membrane with specific primary antibodies (usually diluted in PBST containing the blocking agent). Dilute ACSL4 antibody 1:1000 and GAPDH antibody 1:5000.
8. Washing: Wash the membrane with PBST for 5 minutes each time, for a total of 3 times.
9. Secondary Antibody Incubation: Incubate the membrane with HRP-conjugated secondary antibodies (anti-rabbit: 1:10000 dilution, anti-mouse: 1:10000 dilution). Include washing steps to remove excess secondary antibodies.
10. Detection: Use ECL (enhanced chemiluminescence) substrate to detect the proteins.

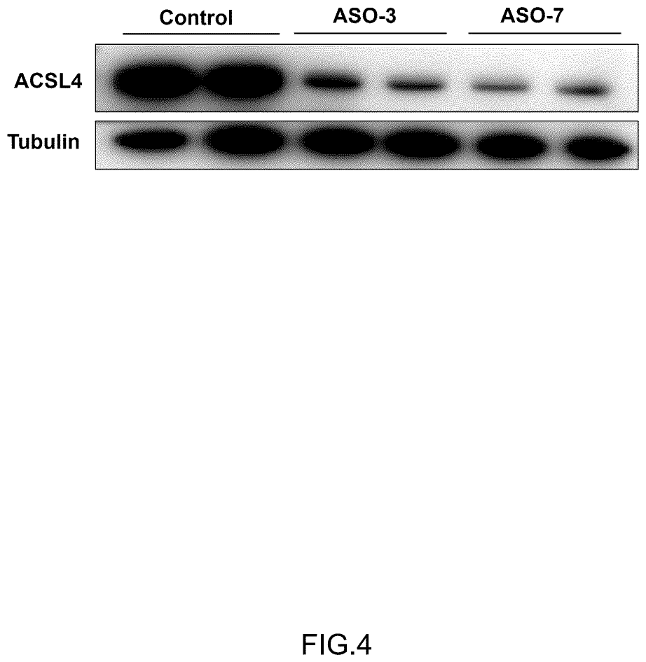

Detection Results: As shown in FIG. 4, ASO-3 and ASO-7 can significantly reduce the expression of ACSL4 protein 36 hours after transfection at 200 nM.

Example 3: Intrathecal Injection (Intrathecal, IT, injection is a method of delivering drugs or compounds directly into the cerebrospinal fluid surrounding the spinal cord. It is commonly used to study the potential effects of drugs on the central nervous system). The following are the specific steps for intrathecally injecting ASO into rats:

### 1. Preparation

ASO and Syringe Preparation: Dilute the ASO to the appropriate concentration as needed and load it into a sterile syringe.

Animal Preparation: Select healthy rats and group them according to the experimental design. Ensure that the animals are fasting before the experiment to reduce the risk of vomiting during anesthesia.

### 2. Anesthesia and Sterilization

Anesthesia: Administer general anesthesia to the rats via intraperitoneal injection using an appropriate anesthetic (such as isoflurane or propofol).

Sterilization: Secure the rats on the operating table, shave the injection site, and clean the skin with 70% alcohol or another sterilizing agent.

### 3. Localization and Injection

1) After Anesthesia: Shave the injection site and disinfect the skin surface with 70% alcohol. With the left thumb and middle finger, gently pull the skin taut over the rat's iliac crests on both sides. Use the index finger to locate the rat's spine at the level of the iliac crests. The highest point touched is the L6 spinous process. Insert a sterile microsyringe vertically into the interspace between the L5 and L6 spinous processes. When a void sensation is felt and the rat's tail or hind limbs show a slight twitch, and a small amount of blood is drawn back, the syringe is confirmed to be in the subarachnoid space.
2) Tilt and Inject: Slowly tilt the syringe from a vertical position to a 45° angle. Inject the ASO based on the rat's body weight. After the injection, retain the syringe for a short period, then slowly withdraw the microsyringe and gently press the injection site for 1-2 minutes.

Note: It may take several attempts to find the correct entry point. There should be no resistance when the needle is inserted into the spine, and a reflexive twitch in the tail or hind limbs indicates that the needle has been correctly placed.

Note: The injection time should be controlled between 10-30 seconds. Injecting too quickly can cause hind limb spasms in the rat, which usually disappear within 60 seconds. After the injection, rotate the needle 180° once or twice, then withdraw it from the spine.

### 4. Observation

Place the rat back in its cage and observe to ensure that normal motor function is restored.

### Example 4: Replication of Rat Middle Cerebral Artery Occlusion/Reperfusion (MCAO/R) Model

The rat middle cerebral artery occlusion/reperfusion (MCAO/R) model is a commonly used animal model for studying cerebral ischemia and reperfusion injury. This model simulates the process of vessel occlusion and subsequent blood flow restoration that occurs during a human stroke. The steps are as follows:

### Preoperative Preparation

1. Anesthesia of Rats: Anesthetize the rats using an appropriate anesthetic (such as isoflurane or pentobarbital).
2. Temperature Maintenance: During the surgery, use a heating pad or lamp to maintain the rat's body temperature.

### Surgical Procedures

3. Incision: Make an incision about 1.5 cm long 0.5 cm to the right of the midline of the rat's neck. Tie off the proximal end of the common carotid artery (CCA) and clamp the distal end with a single vascular clamp. Place a live knot of suture thread on the CCA. Use ophthalmic scissors to make a “V”-shaped incision 1 cm from the bifurcation.
4. Isolation of Vessels: Carefully isolate the vessels using silk thread, taking care to avoid damaging the nerves.
5. Insertion of Suture: Select an appropriate occlusion suture (such as a nylon filament or silicone-coated nylon filament). Insert the suture through the incision, release the arterial clamp on the CCA, and gently pull the ligature on the proximal end of the CCA to the right. Adjust the angle of insertion to the left to guide the suture into the internal carotid artery (ICA). Re-adjust the angle of insertion to approximately 15° to the right and gently pull the CCA to ensure the suture enters the brain, causing focal cerebral ischemia. Leave 1 cm of the suture outside the incision. If the suture encounters difficulty advancing at around 12 mm, it may have entered the posterior communicating artery (PPA). In this case, withdraw the suture a short distance and re-adjust the angle of insertion to the right and up. If multiple attempts fail, it indicates that the ICA may have spasmed. Blood retention can alleviate the spasm; withdraw the suture and wait for 3-5 minutes for blood flow to resume, then attempt to insert the suture again for a higher success rate.
6. Confirmation of MCAO: Insert the suture to a depth of 18-20 mm (starting from the bifurcation of the external carotid artery (ECA) and ICA). Stop when slight resistance is encountered (at this point, the rat's right facial muscles may twitch). Ensure that the tip of the nylon suture passes through the starting point of the middle cerebral artery (MCA) and reaches the finer anterior cerebral artery. This completes the occlusion on one side. Reperfusion
7. Maintenance of Ischemia: Leave the suture in place for 120 minutes to simulate the ischemic state.
8. Removal of Suture: At the predetermined time point after ischemia, slowly pull the nylon filament to retract its tip back into the common carotid artery, thereby achieving reperfusion of the middle cerebral artery.
9. Wound Closure: After reperfusion, carefully suture the neck incision. Maintain the room temperature at 25-30° C. during the ischemia and reperfusion periods.

### Postoperative Management

10. Postoperative Care: Place the rat back in its cage after it has awakened. Provide fresh food and water. Monitor the animal's body temperature, consciousness, and motor function, and promptly address any postoperative complications.
11. Medication: Administer antibiotics and pain relief medication as needed.

neurological scores were assessed using the modified Longa 5-point scale (see Table 2).

TABLE 2
Longa 5 Scale

| Modified Longa 5-Point Scale | scale |
|---|---|
| No neurological deficit symptoms | 0 |
| Mild focal neurological deficit, i.e., the forepaw on the | 1 |
| contralateral side of the surgery does not extend when the rat is | |
| suspended by the tail | |
| Moderate focal neurological deficit, i.e., the rat circles toward the | 2 |
| contralateral side of the surgery when walking | |
| Severe focal neurological deficit, i.e., the rat has difficulty | 3 |
| walking and falls toward the contralateral side of the surgery | |
| Unable to walk spontaneously, decreased level of consciousness | 4 |
| Death | 5 |

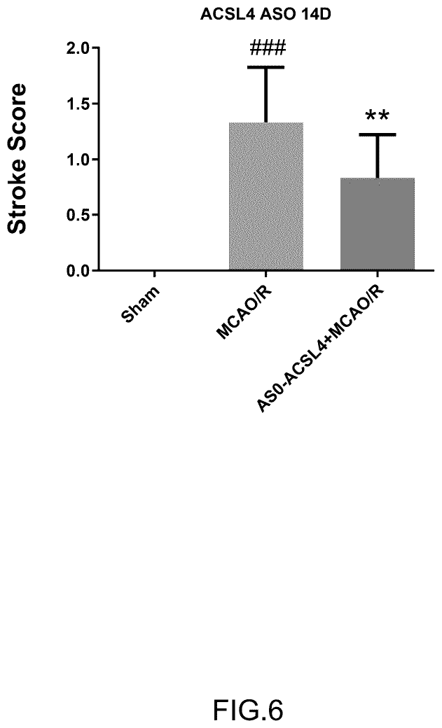

FIG. 6 shows that intrathecal injection of ACSLA-ASO (300 μg per rat) for 14 days significantly improved the neurological behavior of rats in the MCAO (middle cerebral artery occlusion) animal model.

### Example 5: TTC Staining and Infarct Volume Measurement

The MCAO/R (middle cerebral artery occlusion/reperfusion) animal model was constructed as described in Example 4. After the experiment, the rats were anesthetized with 10% chloral hydrate (0.3 ml/100 g) and quickly decapitated to remove the brain. The olfactory bulbs were removed, and the brain was frozen in a −20° C. refrigerator for 10 minutes. After freezing, the brain was removed and sectioned into 5 consecutive coronal slices (each slice approximately 2 mm thick). The brain slices were then placed in a 0.2% 2,3,5-triphenyltetrazolium chloride (TTC) solution and incubated at 37° C. in the dark for 15 minutes. After incubation, the brain slices were transferred to a 4% paraformaldehyde solution and fixed overnight. The brain slices were then removed and photographed using a digital camera to evaluate the infarct condition of the brain tissue.

The infarct area (pink represents normal, undamaged tissue, while the infarct region appears white due to mitochondrial damage) was calculated using the Motic Images Plus 2.0 microscopic image analysis system. Since edema can affect the infarct volume, a correction formula was used. The specific formula is as follows:

$$Infarct Volume ( {mm}^{3} ) = Total Infarct Area \times Slice Thickness ( 2 mm )$$

$$Edema - Corrected Infarct Volume ( {mm}^{3} ) = Infarct Volume \times Volume of the Contralateral Hemisphere / Volume of the Ipsilateral Hemisphere$$

$$Infarct Rate ( \% ) = Edema - Corrected Infarct Volume / Total Volume of Both Hemispheres \times 100 \%$$

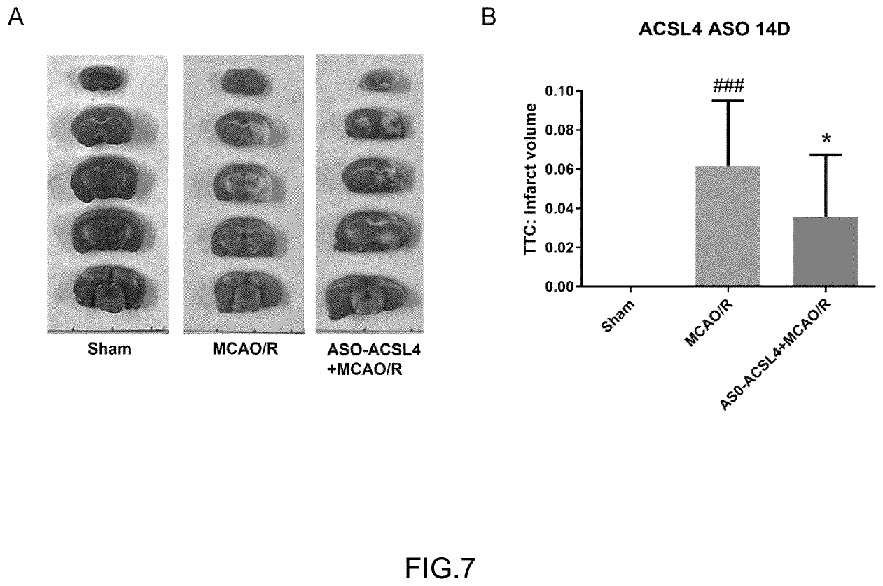

The results shown in FIG. 7 indicate that intrathecal injection of ACSL4-ASO7 can reduce the infarct volume in rats with cerebral ischemia-reperfusion injury.

### Example 6: HE Staining to Detect Infarct Condition, the Steps Are as Follows

1. At various time points after surgery, the rats were anesthetized with chloral hydrate (0.3 mL/100 g), decapitated to remove the brain, and the olfactory bulbs were removed.
2. The brain was frozen at −20° C. for 10 minutes, then removed and sectioned into 5 consecutive coronal slices (each slice approximately 2 mm thick). The 2nd, 3rd, and 4th slices were fixed in 4% paraformaldehyde overnight, placed in a tissue dehydration box, rinsed with running water for 2 hours, and then dehydrated in a tissue processor (see Table 3).

TABLE 3
Brain Tissue Dehydration Steps

| Regents | Time |
|---|---|
| 70% Ethanol | 30 min |
| 80% Ethanol | 30 min |
| 90% Ethanol | 20 min |
| 95% Ethanol | 20 min |
| Ethanol 1 | 20 min |
| Ethanol 2 | 20 min |
| Xylene 1 | 15 min |
| Xylene 2 | 15 min |
| Xylene 3 | 20 min |
| Paraffin 1 | 30 min |
| Paraffin 2 | 30 min |
| Paraffin 3 | 1 h |

After dehydration, the brain tissue was embedded using a paraffin embedding machine. The embedded tissue was cooled and sectioned to a thickness of 5 μm, then dried at 37° C. and stored for future use. The paraffin sections were baked in an oven for 30 minutes, removed from the oven, and subjected to HE staining (see Table 4).

They were then sealed with neutral gum and photographed.

TABLE 4
HE staining

| Reagents | Time | |
|---|---|---|
| Xylene 1 | 10 | min |
| Xylene 2 | 10 | min |
| Ethanol 1 | 3 | min |
| Ethanol 2 | 3 | min |
| 95% Ethanol | 2 | min |
| 90% Ethanol | 2 | min |
| 80% Ethanol | 1 | min |
| 70% Ethanol | 1 | min |
| Water | 5 | min |
| Hematoxylin | 5 | min |
| Water wash | 5 | min |
| 1% Hcl | 2-3 | s |
| Water wash | 2 | min |
| Eosin | 45 | s |
| 80% Ethanol | 2-3 | s |
| 90% Ethanol | 2-3 | s |
| Ethanol 1 | 3 | min |
| Ethanol 2 | 3 | min |
| Xylene 1 | 10 | min |
| Xylene 2 | 10 | min |

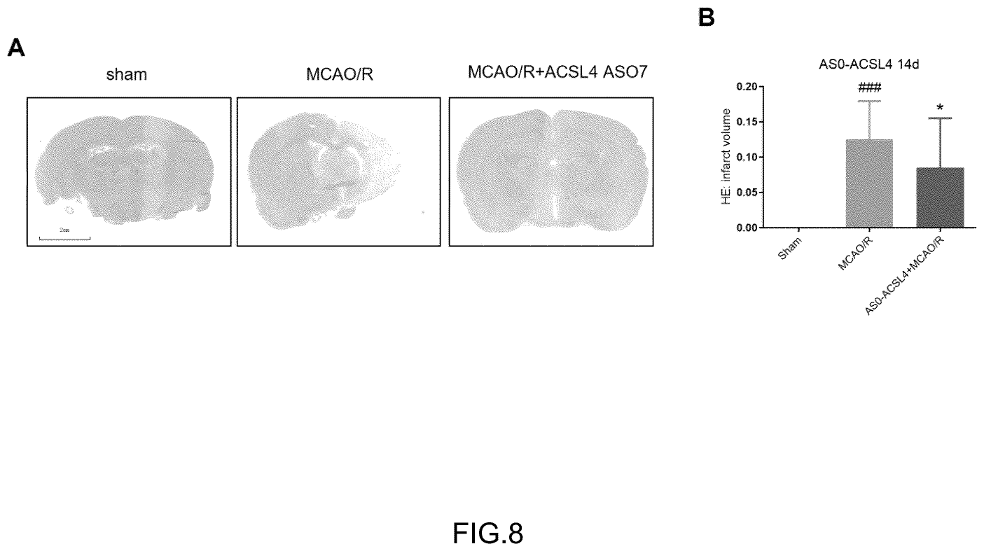

The final result is shown in FIG. 8, which indicates that ACSLA-ASO7 injection can reduce the infarct volume in the brains of rats with cerebral ischemia-reperfusion injury.

The materials used in this experiment are as follows:
1. Human embryonic kidney cells (human HEK293 cells); Rats, SD, male, SPF grade, supplier: Sparrow (Beijing) Biotechnology Co., Ltd.; Suture thread: Beijing Senion Technology Co., Ltd., model: 2636A2.
2. Serum-free medium for human cells: DMEM medium (company: HyClone, catalog number: 11965126).
3. Opti-MEM medium (company: Thermo Fisher Scientific, catalog number: 11058021); Celopener transfection reagent (company: Shanghai Jifluo, catalog number: 13778150).
4. TRIZOL (company: Invitrogen (Shanghai), catalog number: 15596026); Hieff® qPCR SYBR® Green Master Mix (company: Bioclor, catalog number: 11203ES08); First-strand cDNA synthesis kit (company: Thermo Fisher Scientific, catalog number: K1622).
5. Antibodies: Anti-ACSL4 antibody (company: Abcam, catalog number: ab1002422-15); Anti-beta-tubulin mouse antibody (company: Kangwei Century Biotechnology Co., Ltd., catalog number: 01270/07223).

The embodiments of the present invention are provided for illustrative and descriptive purposes. Although embodiments of the invention have been shown and described, it is to be understood that these embodiments are illustrative and should not be construed as limiting the invention. Ordinary skilled persons in the art can make various changes, modifications, substitutions, and variations to the embodiments within the scope of the present invention.

What is claimed is:

1. An antisense oligonucleotide for inhibiting ACSL4 gene expression, characterized in that the oligonucleotide sequence is as shown in SEQ ID NO. 3 and SEQ ID NO. 7.


SEQUENCE LISTING (10 sequences)

SEQ ID NO: 1 - DNA, 20 bp, synthetic construct
  source 1..20: mol_type = other DNA
tgtactcaccggaacagcag

SEQ ID NO: 2 - DNA, 20 bp, synthetic construct
  source 1..20: mol_type = other DNA
gtactcaccggaacagcagc

SEQ ID NO: 3 - DNA, 20 bp, synthetic construct
  source 1..20: mol_type = other DNA
tgggcttcagtacagtacag

SEQ ID NO: 4 - DNA, 20 bp, synthetic construct
  source 1..20: mol_type = other DNA
ttcagtacagtacagtctcc

SEQ ID NO: 5 - DNA, 20 bp, synthetic construct
  source 1..20: mol_type = other DNA
tactcaccggaacagcagcc

SEQ ID NO: 6 - DNA, 20 bp, synthetic construct
  source 1..20: mol_type = other DNA
tcccagcaccacatgattct

SEQ ID NO: 7 - DNA, 20 bp, synthetic construct
  source 1..20: mol_type = other DNA
gcagacattcatgaatcggt

SEQ ID NO: 8 - DNA, 20 bp, synthetic construct
  source 1..20: mol_type = other DNA
ccagcaccacatgattctgt

SEQ ID NO: 9 - DNA, 20 bp, synthetic construct
  source 1..20: mol_type = other DNA
cagacattcatgaatcggtg

SEQ ID NO: 10 - DNA, 20 bp, synthetic construct
  source 1..20: mol_type = other DNA
cattcatgaatcggtgtgtc